(19) United States
(12) Patent Application Publication
(10) Pub. No.: US 2010/0082042 A1
(43) Pub. Date: Apr. 1, 2010

(54) BIOLOGICAL UNIT REMOVAL TOOL WITH OCCLUDING MEMBER

(76) Inventor: Michael J. Drews, Palo Alto, CA (US)

Correspondence Address: RESTORATION ROBOTICS, INC., 1383 SHOREBIRD WAY, MOUNTAIN VIEW, CA, 94043, US

(21) Appl. No.: 12/558,102

(22) Filed: Sep. 11, 2009

Related U.S. Application Data
(60) Provisional application No. 61/101,544, filed on Sep. 30, 2008.

(51) Int. Cl. A61B 19/00; A61B 17/50

(52) U.S. Cl. 606/130; 606/133

(57) ABSTRACT

Tools and methods are provided for removing biological units from a body surface utilizing a removal tool. The tools incorporate occluding members to help retain the biological unit in the removal tool and assist in severing the biological unit from the surrounding connective tissue. The occluding member may be located at a distal end of the tool and close in an iris configuration over the distal tool tip or the occluding member may constrict along the tool midsection. The occluding member may be an elastomeric sleeve or it could comprise filaments that are arranged to constrict upon being twisted or rotated. The tools are especially useful for removing follicular units from a body surface in a hair transplantation process, and especially in the context of a robotic system.

### RELATED APPLICATIONS

This application claims priority under 35 U.S.C. 119(e) to U.S. Provisional Application No. 61/101,544 filed Sep. 30, 2008, entitled “BIOLOGICAL UNIT REMOVAL TOOL WITH OCCLUDING MEMBER”.

### FIELD OF THE INVENTION

This invention relates generally to tools used for the harvesting of various biological tissue samples, in particular hair follicles.

### BACKGROUND OF THE INVENTION

There are various known tools and instruments for removing biological tissue samples from the body. Biopsy needles and punches are used when a small tissue specimen is required for examination, for example, to identify certain medical conditions. Another example of the biological tissue which is often desired to be removed or harvested is a hair follicle. Hair transplantation procedures are well-known, and typically involve harvesting donor hair grafts from the “donor areas,” for example, side and back fringe areas of the patient's scalp, and implanting them in a bald area (“recipient area”). Historically, the harvested hair grafts were relatively large (3-5 mm), although more recently the donor grafts may be single “follicular units,” which are naturally occurring aggregates of 1-3 (and much less commonly, 4-5) closely spaced hair follicles that are distributed randomly over the surface of the scalp. In one well-known process, a linear portion of the scalp is removed from a donor area by dissection, using a scalpel to cut down into the fatty subcutaneous tissue. The strip is then dissected (under a microscope) into the component follicular units, which are then implanted into a recipient area in respective puncture incisions made by a needle or razor blade. Forceps are typically used to grasp and place the follicular unit grafts into the needle puncture locations, although other instruments and methods are known for doing so.

For instance, U.S. Pat. No. 7,172,604 (Cole) discloses an instrument for the extraction of individual follicular units. U.S. Patent Publication 20050267506 (Harris) discloses a method and apparatus for the extraction of follicular units by first scoring the outer skin layers with a sharp punch, and then inserting a blunt punch into the incision to separate the hair follicular unit from the surrounding tissue and fatty layer to reduce the incidence of hair transection. Another U.S. Pat. No. 6,585,746 (Gildenberg) discloses a hair transplantation system utilizing a robotic system, including a robotic arm and a hair follicle end effector associated with the robotic arm that could be used to harvest hair follicles from the donor area.

Despite certain advances in improving the tools for harvesting of biological tissue, there remains a need for a more efficient harvesting tool that increases the yield of usable harvested specimens, improves retention of the harvested units in the removal tool and the quality of the obtained specimens.

### SUMMARY OF THE INVENTION

The present disclosure provides a number of solutions to deficiencies in the prior art and includes various features for increasing the yield of usable harvested biological specimens for instance a follicular unit, a skin sample, a tissue sample, or a biopsy unit. In general the invention provides tools that effectively penetrate tissue and remove and retain biological units therein without damaging them. One particularly useful application for the tools described herein is in the area of hair harvesting and transplantation, which requires the removal of countless follicular units. The tools can be manually operated or incorporated into an automated system, including robotic system.

In one embodiment a biological tissue removal tool comprises an elongated body and an occluding member disposed coaxially with the elongated body. The elongated body has a lumen sized to receive a biological unit and a distal tip configured to penetrate a body surface. The occluding member has a first end and a second end, a first configuration where the lumen of the elongated body is substantially open, and a second configuration wherein at least a portion of the occluding member partially or fully occludes the lumen of the elongated body, and wherein in the second configuration, at least one of the first end or the second end of the occluding member is rotated relative to the other end. In some embodiments, either the first end or the second end, or both ends, of the occluding member may be attached to the elongated body, for example, at the respective first and/or second ends of the elongated body, or to its exterior. The tool may be a hair harvesting tool and the biological unit is a follicular unit. The tool may further comprise a mechanism for controlled rotation of the occluding member, and/or controlled tension of the occluding member, and/or an overload protection mechanism for the occluding member. Also, the tool may have a tapered portion at its distal end.

The elongated body may comprise a first tube, and the tool further includes a second tube, wherein the occluding member may connect at least at one end to such second tube. The occluding member may also connect at its other end to the first tube. The first and second tubes may be concentrically arranged as inner and outer tubes, respectively, and the occluding member connects to the exterior of both tubes. The occluding member in the second configuration may constrict over the distal tip of the inner tube.

In another embodiment, the occluding member comprises one or more of a flexible sleeve, a slot (e.g. one or more helical slots), or a filament. If the occluding member is a flexible sleeve, it may be attached on a first end to a thinned segment proximal to the distal tip of the inner tube and on a second end to a thinned region on the outer tube, and the first and second ends extend in a proximal direction such that the occluding member folds back upon itself and forms a rolling fold at a distal end thereof that can be advanced beyond and constrict over the distal tip of the inner tube. In certain embodiments, the occluding member may constrict or occlude the lumen of the elongated body proximally away from the distal tip of the elongated body. For instance, in one version the first and second tubes are co-linear and spaced apart across an axial gap, and the occluding member in the second configuration constricts into the lumen through the axial gap.

In another aspect, the occluding member comprises at least one filament with a first end fixed with respect to the elongated body and a second end configured to rotate around the elongated body. The filament is arranged to transition upon rotation around the elongated body from a position in the first configuration generally outside of the lumen of the elongated body to a position in the second configuration where at least a portion of the filament extends across the lumen at the distal tip of the elongated body. The tool may also include one or more channels structured to accommodate the at least one filament in the first configuration. The occluding member may comprise a plurality of filaments each with the first and second ends, wherein in the second configuration the filaments extend across the lumen at the distal tip of the elongated body in an overlapping fashion. Furthermore, in some embodiments, the occluding member, or at least an occluding portion of the occluding member, may comprise one or more slots, such as helical slots. In some embodiments at least one end of the occluding member may be fixed, for example, with respect to the elongated body or with respect to another co-axial member. Upon relative rotation, for example, of at least the occluding portion of the occluding member and the elongated body, the lumen of the elongated body may be occluded at least at the occluding portion.

Another aspect of the invention is a biological tissue removal tool, for example, a follicular unit harvesting tool, comprising an elongated body having a lumen sized to receive a biological unit, such as follicular unit, and a distal tip configured to penetrate a body surface. A co-axial member mounts on the elongated body, and an occluding member has a first end attached to the elongated body and a second end attached to the co-axial member. The occluding member is configured to at least partially occlude or close the lumen of the elongated body upon relative axial and/or rotational movement of the elongated body and the co-axial member.

The occluding member may comprise at least one filament with a first end and a second end, the at least one filament being arranged to transition upon relative rotation of the co-axial member and the elongated body from a position substantially adjacent the elongated body to a position where a portion of the filament extends across the distal tip of the elongated body. The occluding member may comprise a plurality of filaments, wherein in an occluding position the filaments extend across the distal tip of the elongated body in an overlapping fashion. The tool may include an overload protection mechanism for the occluding member. In some embodiments, the occluding member may comprise one or more filaments extendable over the distal end of the elongated body and either the elongated body or the co-axial member may include one or more channels in which the one or more filaments reside prior to relative rotation of the elongated body and the co-axial member.

Alternatively, the elongated body comprises two co-axial inner and outer harvesting cannulas, and the co-axial member is mounted on the outer harvesting cannula. The co-axial member preferably comprises a second elongated body axially movable with respect to the first elongated body, and the occluding member comprises a tubular member connected to distal ends of both the first and second elongated bodies. The tubular occluding member may be folded back upon itself and forms a rolling fold at a distal end thereof that can be advanced beyond a distal tip of the first elongated body. The tubular member may be a flexible sleeve.

A method of removing biological tissue, for example, a hair follicle or a follicular unit, from a donor area is disclosed herein. The method may comprise advancing a removal tool to penetrate a donor area and surround a biological unit to be removed or harvested. The removal tool includes an elongated body having a lumen sized to receive such biological unit, a distal tip configured to penetrate tissue, and an occluding member coaxially disposed relative to the elongated body and having a first end and a second end. The method also includes rotating either the first end, or the second end, or both ends of the occluding member relative to the other end or relative to each other to convert the occluding member from a first configuration where the lumen of the elongated body is substantially open to a second configuration where at least a portion of the occluding member occludes the lumen of the elongated body. The method also comprises withdrawing the removal tool to remove the biological unit from the donor area with the assistance of the occluding member.

Furthermore, harvesting biological unit, such as the follicular unit, may comprise robotically assisted harvesting. In one embodiment, the step of advancing the elongated body to penetrate the donor area and surround a biological unit comprises extending a distal tip of the elongated body past a bulb of the follicular unit. Alternatively, the step of advancing comprises advancing a distal tip of the elongated body to a position along the length of the follicular unit, and the step of rotating causes the occluding member to intimately engage the follicular unit without severing it.

According to another aspect of the invention, a further method of removing biological tissue from a donor area is provided. The method may include positioning a removal tool adjacent a donor area. The removal tool includes a first elongated body having a lumen sized to receive a biological unit and a distal tip adapted to penetrate tissue, a second elongated body, wherein the first elongated body and the second elongated body are concentrically movable relative to each other, and an occluding member having a first end fixed with respect to the first elongated body and a second end fixed with respect to the second elongated body. The method also includes the steps of advancing the first elongated body to penetrate the donor area and surround a biological unit; relatively rotating the first and/or the second elongated bodies to convert the occluding member from a first configuration where the lumen of the first elongated body is substantially open to a second configuration where at least a portion of the occluding member occludes the lumen of the first elongated body; and withdrawing the first and second elongated bodies to remove the biological unit from the donor area with the assistance of the occluding member.

The method may include axially advancing the second elongated body relative to the first elongated body so that a distal tip of the second elongated body extends around the biological unit. In one embodiment, the occluding member is a flexible sleeve extending over and distally beyond the second elongated body and is folded back to be fixed to the first elongated body. If the method includes axially advancing the second elongated body relative to the first elongated body, the fold of the occluding member advances forward. Alternatively, the method may feature introducing a gas or fluid into the removal tool to expand and distally advance the occluding member. For example, the gas or fluid may be introduced into a space between the first and second elongated bodies. Desirably, the biological unit is a follicular unit, and withdrawing the first and the second elongated bodies comprises harvesting a follicular unit from the donor area.

The occluding member may comprise a filament fixed to the first and second elongated bodies and extending over a distal tip of the first elongated body. The filament may reside in a channel or recess prior to the step of relatively rotating the first and the second elongated bodies. The method may further include adjustably relatively rotating the first and the second elongated bodies depending on a desired amount of occlusion of the lumen of the first elongated body by the occluding member.

According to another aspect of the invention, an automated system for harvesting follicular units is provided. In one embodiment, such automated system comprises a moveable arm, a hair harvesting tool operably connected to the moveable arm and a control mechanism for controlling movements of one or more of the moveable arm and/or the harvesting tool. The harvesting tool includes an elongated body having a lumen sized to receive a biological unit and a distal tip configured to penetrate a body surface; and an occluding member disposed coaxially with the elongated body and having a first end and a second end, the occluding member having a first configuration where the lumen of the elongated body is substantially open, and a second configuration where at least a portion of the occluding member partially or fully occludes the lumen of the elongated body, wherein in the second configuration, at least one of the first end or the second end of the occluding member is rotated relative to the other.

It should be understood that the various features of the removal tools described in reference to one of the embodiments may be combined and used with other features described in reference to other embodiments described herein unless expressly mutually exclusive. For example, one or more constriction features can be combined with various described distal tips, such as those having cutting and relief segments.

Other and further objects and advantages of the invention will become apparent from the following detailed description when read in view of the accompanying figures.

### BRIEF DESCRIPTION OF THE DRAWINGS

Features and advantages of the present invention will become appreciated as the same become better understood with reference to the specification, claims, and appended drawings wherein:

FIGS. 1-4 are perspective views of the operation of one example of an embodiment of the biological unit removal tool of the present invention having a single filament;

FIGS. 5-8 are perspective views of the operation of an alternative removal tool having two diametrically opposed filaments;

FIGS. 9-12B are perspective views of the operation of additional removal tools having three or more circumferentially spaced filaments;

FIGS. 13-16 are perspective views of the operation of a further example of an embodiment of the biological unit removal tool of the present invention having a sleeve-like occluding member;

FIGS. 17-22A are side and longitudinal sectional views of the biological unit removal tool of FIGS. 13-16 including some variations;

FIGS. 23 and 24A-24E are perspective, elevational, and sectional views of an example of a hand-held system for operating a biological unit removal tool similar to that shown in FIGS. 13-22;

FIG. 25 is a schematic perspective view of an example of a robotic system for operating biological unit removal tools of the present invention;

FIGS. 26A-26C illustrate a distal end of a robotic system showing operation of an example of a dual cannula biological unit removal tool;

FIGS. 27-31 are longitudinal sectional views of the biological unit removal tool of FIGS. 13-22 during different stages of operation of removing a follicular unit from a body surface;

FIGS. 32A and 32B are elevational views of another alternative removal tool having an elastomeric sleeve occluding member positioned between two co-linear tubular members;

FIGS. 33A and 33B are elevational views of a further alternative removal tool having a fibrous occluding member positioned to constrict inward through axial gaps between two co-linear tubular members; and

FIGS. 34A and 34B are partial sectional views of another alternative removal tool having an occluding member.

FIG. 35A-35D are various views of another alternative removal tool with helical openings in the occluding member.

### DETAILED DESCRIPTION OF THE PREFERRED EMBODIMENTS

In the following Detailed Description, reference is made to the accompanying drawings that show by way of illustration some examples of embodiments in which the invention may be practiced. In this regard, directional terminology, such as “top,” “bottom,” “front,” “back,” “distal,” “proximal,” etc., is used with reference to the orientation of the Figure(s) being described. Because components or embodiments of the present invention can be positioned in a number of different orientations, the directional terminology is used for purposes of illustration and is in no way limiting. It is to be understood that other embodiments may be utilized and structural or logical changes may be made without departing from the scope of the present invention. The following description, therefore, is not to be taken in a limiting sense, and the scope of the present invention is defined by the appended claims.

The adjective “automated” with reference to a system or process as a whole means that some part or all of a particular system or step in the process involves an autonomous mechanism or function; i.e., that mechanism or function does not require manual actuation. Ultimately, one or more steps in the procedure may be automated, or autonomous, with some parts requiring manual input. This definition encompasses an automated system that requires only an operator to depress an ON switch or schedule the operation, and also a system in which hand held tools are used but some mechanism of the system functions autonomously, i.e., without human input, to perform a function. Some of the automated systems described herein may also be robotically-assisted or computer/software/machine-instruction controlled. The devices and methods of the present invention are useful in manual procedures and systems, as well as in automated procedures and system. The tools of the present invention could be used with the robotically-assisted systems and procedures. The adverb “automatically” when referring to use of a particular component of a system or a particular step in a process means that such step is accomplished autonomously, i.e., without real-time manual assistance.

The term “tool” or “biological unit removal tool” as used herein refers to any number of tools or end effectors that are capable of removing or harvesting various biological tissues, for example, follicular units (“FUs”) from a body surface. In general, however, the tools of the present invention may be useful for removing biological units other than FUs from a body surface. In this sense, a body surface can be attached to the body or may be a flap of skin or body tissue removed from the body. Such tools may have many different forms and configurations. In many embodiments, the tool comprises a hollow tubular shaft and thus may be labeled, for example, a cannula, a needle, or a punch. The distal end of removal tools (for example, punches, coring devices, cutting and/or trimming devices, needles), are typically sharpened, to cut and extract the tissue (e.g., hair follicle).

Various embodiments of follicular unit harvesting cannulas (or tools) described herein may be employed in harvesting systems, whether such systems are fully-automated (e.g., robotically controlled), semi-automated, or manually controlled. It will be appreciated by those skilled in the art that each harvesting cannula design may have certain benefits (e.g., superior retraction and retention of follicular units, less trauma to the surrounding skin and tissue), or drawbacks (e.g., complex design and/or operation, higher manufacturing costs, increased trauma), relative to the other embodiments. Thus, selection of a particular harvesting cannula distal end design will depend on the particular performance criteria sought to be achieved.

“Biological units” include discrete units used in cosmetic, diagnostic, and dermatological procedures, for example, various tissues, including that extracted for biopsies or grafting, fat units, skin units, etc. Examples of the biological units particularly useful with the present invention are hair grafts, or follicles, or “follicular unit(s).” Other biological units may be tissue used for diagnosis of cancer, such as from the areas of the breast, liver, prostate, colon and small bowel, or lungs. Other tissue examples where biopsies are performed include bone, heart and brain tissue. Furthermore, “biological unit” may alternatively be referred to as “biopsy sample,” “biopsy specimen” “biological tissue sample,” or “biological tissue specimen.”

As mentioned above, the term biological units encompasses a number of things, though the present invention is particularly useful in hair harvesting, to provide devices and methods for harvesting follicular units (FUs). As such, the term follicular units (or FUs) will be used herein simply as an example for purposes of describing some embodiments with the understanding that it represents more broadly biological units.

The present invention provides biological unit removal tools, such as follicular unit harvesting tools, with occluding members. Usually, the removal tools have a tubular elongated body with a cylindrical profile and a hollow lumen therethrough, although these tools don't have to be tubular and the profile may be other than cylindrical (e.g., curved and not straight, or other than circular in section). Furthermore, although a particularly useful biological removal tool includes a hollow lumen that extends through the elongated body from one end to another, it is also possible that the lumen only extends part way along the length of the elongated body. More particularly, suction or vacuum is typically used with the biological removal tools described herein, and suction may be created through a lumen that extends the entire length of the elongated body, or in a lumen that only extends part of the way along the body. Various “occluding members” described herein may be positioned not only at the distal end of the tool, but also in various locations along the body of the tool, for example, a short distance from the distal end of the tool, or midway along the body of the tool. Also, the occluding member may be positioned, for example, within a lumen or on the outside of the body. The terms “coupled,” or “attached,” or “connected,” or “mounted” as used herein, may mean directly or indirectly coupled, attached, integrated, or mounted through one or more intervening components.

An “occluding member” as used herein refers to a number of structures that partially or fully block a lumen of various biological removal tools. The term occlude in this sense means to at least partially blocking passage through or otherwise interfering with or obstructing the path of a lumen. As will be seen, the occluding members may constrict about any part of a length of the lumen, or across a distal end of the lumen, or across a circumference of a lumen proximally from the distal end of the lumen. The occluding members may translate into or across the lumen, or radially constrict the lumen in a circumferential manner. To reiterate, “occlude” should not necessarily infer complete blockage or obstruction, but also means partial blockage or obstruction, for example, simply closing tightly about a biological unit, such as follicular unit, located in the lumen to improve its retention and removal without damaging it.

The occluding members described herein may be made of a variety of biocompatible materials, such as polypropylene, polyester, polyurethane, Teflon, Nitinol, stainless steel, etc. The configuration of the occluding members may be solid, braided, filamentous, etc., as will be described, and should not be considered limited to any one particular embodiment.

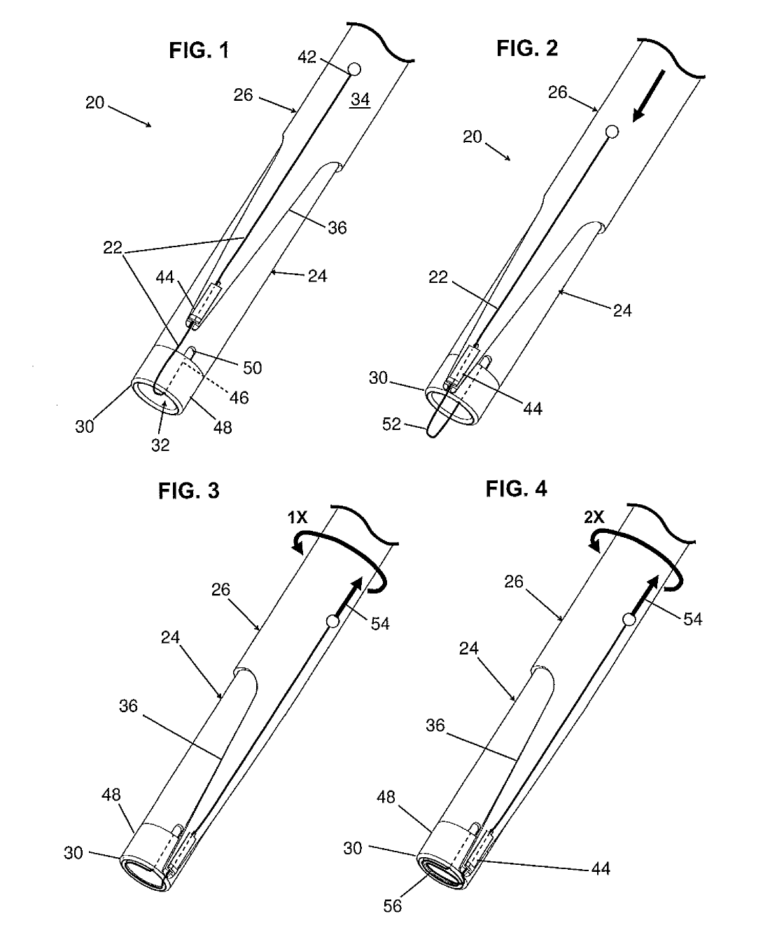

FIGS. 1-4 illustrate a first example of an embodiment of the biological unit removal tool 20 having a single filament 22 connected with respect to an elongated body or a tube 24 and co-axial member 26 for facilitating detachment of a biological unit held within the tool from the surrounding connective tissue. The tool 20 is generally tubular and defines a distal tip 30 forming a distal opening of an inner lumen 32. The removal tool 20 may be used to remove or harvest a plurality of different biological units, and will be correspondingly sized. For example, in one embodiment, a follicular unit removal tool 20 having an inner lumen 32 with a diameter of between about 0.5 to 1.5 mm. Only the distal end of the tool 20 is shown, the length being variable depending on the system in which it is used. The tool 20 may have a length of between about 4 to 25 mm depending on the application.

In the embodiment depicted in FIGS. 1-4, the inner cannula or tube 24 comprises an elongated body that extends the length of the tool 20. The outer tube or co-axial member 26 may have a cylindrical profile on a proximal end 34, but reduces to a single tapered finger 36 on a distal end. Although not shown, the member 26 is mounted for relative axial and rotational motion with respect to the inner elongated body 24. Generally speaking, the various ways to accomplish relative axial and rotational motion between the tube 24 and the member 26 can be described as a first tube or member (inner or outer) being mounted for relative rotation and axial movement with respect to a second tube or member (inner or outer). Finally, the outer tube 26 may be replaced with various co-axial members mounted for rotation about the elongated body 24. For instance, the co-axial member 26 may include the tapered finger 36 on its distal end, and is not entirely tubular. A similar result could be obtained with the co-axial member 26 that simply consists of an elongated finger, as long as it is arranged to move coaxially with respect to the inner tube.

Furthermore, the present application provides occluding members for biological unit removal tools that comprise a single cannula or tube 24 as shown in FIGS. 1-4, or dual cannulas (not shown) for combined relatively sharp and dull dual coring of the relevant object, such as a follicular unit. Such dual coring cannulas are described in the commonly owned patent application Ser. No. 12/050,917, filed on Mar. 18, 2008, which is incorporated herein by reference in its entirety. A single cannula removal tool may include movable or stationary retention features therein. The dual cannula removal tool includes an inner cannula concentrically surrounded by an outer cannula. Typically, one of the dual cannulas has a sharp distal tip and is first advanced into the body surface followed by a blunter second cannula. An occluding member may be operatively mounted on one or both of these concentric dual cannulas. To this extend, the term “elongated body” refers to any of the aforementioned single or dual cannula removal tools, each having a lumen sized to receive a biological unit and a distal end configured to penetrate a body surface.

A filament 22 is shown in FIGS. 1-4 to be fixed at a first end 42 to a point on the co-axial member 26 and passes through a guide 44 on the distal tip of the tapered finger 36. The filament 22 also has a second end 46 that is connected to the tube or elongated body 24. More particularly, in one example of the attachment of the second end 46 of the filament 22 to the tube 24, a distal sleeve 48 may non-rotatably mount around the distal end of the tube 24, and the filament 22 extends around the distal tip 30 and turns 180° proximally into a concentric gap between the tube 24 and sleeve 48. The second end 46 of the filament 22 affixes to a point either within this gap or at the proximal end of sleeve 48. For example, the filament 22 may have a small bead (not shown) on its second end 46 that fits closely within a cut out 50 in the sleeve 48. Alternatively, the filament 22 may also be affixed by means of an adhesive, ultrasonic welding, overmolding, or the like. Those of skill in the art will understand that there are various ways of attaching the filament 22 to the tube 24.

In the configuration illustrated, the guide 44 protrudes slightly from the surface of the elongated body 24. However, it may be desirable for the guide 44 to keep a low profile with respect to the proximal end of sleeve 48. In so doing, the transition from the proximal end of sleeve 48 to the distal tip of the tapered finger 36 to be minimized, easing operation of the removal tool 20 at it is advanced to puncture a body surface. This may be accomplished by slightly tapering the elongated body 24 such that its distal end is of a smaller diameter than its proximal end. Alternatively the distal end can be stepped, reduced in thickness or otherwise adapted to minimize this transition.

The single filament 22 may comprise a suture, a thread, a strand, a string, a fiber, a wire, a cable, or a yarn, it may be comprised of a mono-filament or from multi-filaments, either braided or merely axially aligned, or the like. Whether a single filament is a mono-filament or multi-filament, it may be coated, impregnated or overmolded for protection, for example, with silicone, epoxy, Pebax®, or other appropriate material. In some embodiments, multi-filaments may be encapsulated for protection into a wire coil jacket. The filament made be made from a metal, for example stainless steel, nickel-titanium alloys, or the like, or from a polymer or polymers, for example, polyester, ultra-high molecular weight polyethylene (UHMWPE), polyethylene, polytetrafluoroethylene (PTFE), expanded polytetrafluoroethylene (ePTFE), aramid fibers like Kevlar® (Dupont, Wilmington, Del.), or the like. Furthermore, the surface of the filament 22 can be smooth or can be grooved, ridged, roughened, coated, clad, or modified in any way to provide additional texture to further enhance cutting.

The single filament 22 is arranged to transition upon relative rotation of the tube 24 and the co-axial member 26 from a first position where the lumen 30 of the elongated body 24 is substantially open to a position that occludes or at least partially closes the lumen 30 of the elongated body 24. For example, in a constricted configuration, at least a portion of the filament extends across the distal tip 30. The transition between the two positions is shown in the sequence of FIGS. 1-4.

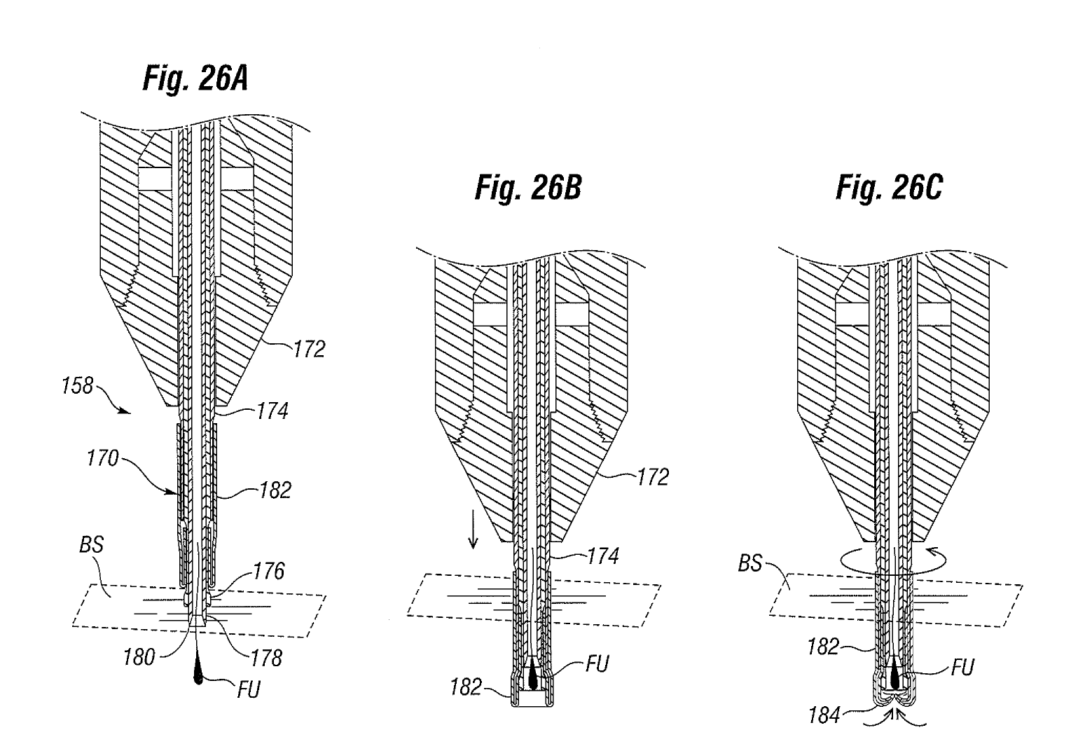

FIG. 1 illustrates an initial configuration wherein the removal tool 20 may be advanced to puncture a body surface and receive a biological unit, such as follicular unit (“FU”), within the lumen 32. The body surface and biological unit are not shown in these figures for clarity, but the reader can reference FIGS. 26-31 for views of alternative devices used for removing or harvesting a follicular unit from a body surface. In the initial configuration, the tapered finger 36 of the co-axial member or tube 26 is spaced proximally from the distal tip 30 and is rotationally aligned with the second end 46 of the filament 22. In this position, the filament 22 tracks along the outside of the tube 24 and loops around the distal tip 30 to affix at the second end 46. Although the filament 22 appears somewhat loose for descriptive purposes, it preferably extends in a taut line from the guide 44 to the second end 46, and therefore extends adjacent to the tube 24.

FIG. 2 shows the co-axial member 26 advanced distally over the tube 24 until the guide 44 lies adjacent the distal tip 30. This movement creates slack 52 in the filament 22 so that the filament extends beyond the distal tip 30. Next, FIGS. 3 and 4 illustrate one (1×) and two (2×) complete rotations of the tube/co-axial member 26 over the tube/elongated body 24. At the same time, tension is applied to the filament 22 as indicated by the arrow 54. Because the filament 22 is held circumferentially at the distal tip 30 by interaction with the tube 24 and sleeve 48, and because of the presence of biological tissue (not shown) in the lumen 32, the slack 52 transitions into first one and then two loops 56. The tube 26 may be rotated once as shown in FIG. 3, or twice as shown in FIG. 4. In some embodiments, it could be further rotated if desired and appropriate under circumstances.

Because of tension in the filament 22, the two loops 56 tend to constrict about the tissue circumscribed by the distal tip 30. That is, the filament 22 undergoes a transition between lying outside of the lumen 32, and constricting into the loops 56 in the lumen. The filament 22 may cut into the tissue somewhat, and further tension applied to the filament may cut all the way through the tissue separating, for example, a follicular unit that is harvested from the connective tissue that holds it back. In certain embodiments, a preferred technique is to cause the loops 56 to constrict into the tissue at the distal tip 30, thus forming a lasso of sorts.

Subsequently, the user retracts the entire tool 20 so as to disengage the biological unit from its surrounding connective tissue. Suction within the lumen 32 assists this removal. Because the filament 22 attaches at both ends to the tool 20, the constricted loops 56 transfer a substantial pulling force to the tissue at the distal tip 30, and facilitate severance of the biological unit. Furthermore, the loops 56 may cut or notch the tissue a small amount, which creates a point of weakness from which a tear in the tissue can easily propagate.

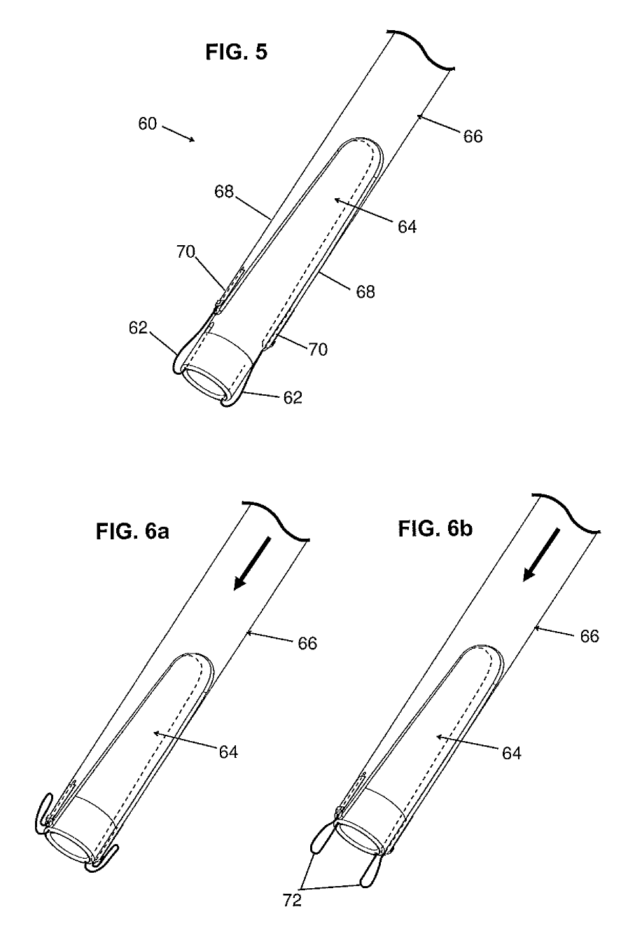

FIGS. 5-8 illustrate the operation of an alternative tubular biological unit removal tool 60 having two diametrically opposed filaments 62, each fixed at respective ends to a tube or elongated body 64 and a co-axial member 66. The removal tool 60 is constructed and functions much like the removal tool 20 described above, though the tube or co-axial member 66 includes two tapered fingers 68 having tips 70 to which the filaments 62 affix. Each of the filaments 62 attaches at a first end to the tube 66 finger tips 70, and at a second end to the tube 64, in the same manner as the earlier embodiment. As before, the tube 66 is mounted for relative axial and rotational movement with respect to the tube 64.

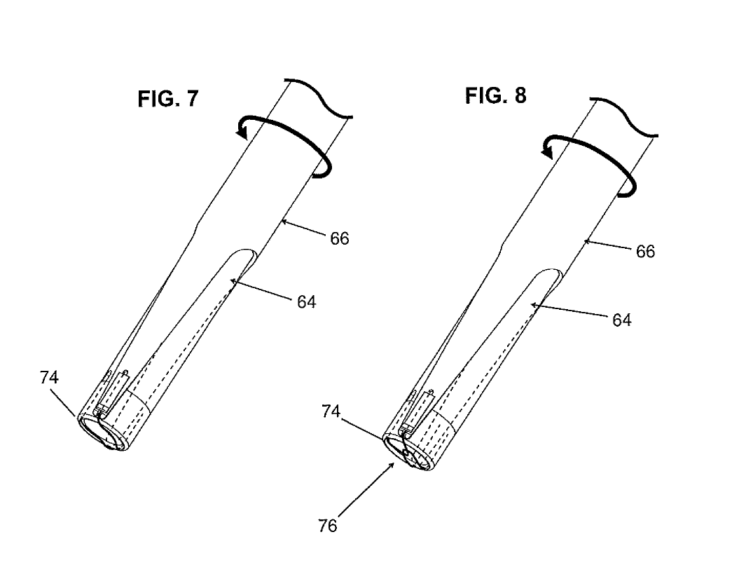

FIGS. 6-8 illustrate the axial advancement and rotation of the tube 66 over the tube 64. FIG. 6b shows the configuration of slack 72 in the filaments 62 past a distal tip 74. FIG. 7 shows the outer tube 66 rotated about 180° relative to the inner tube 64, while FIG. 8 shows the configuration after about 360° rotation. The filaments 62 cross over one another and the resulting mutual tension pulls each filament toward the center of the lumen of the tube 64. The cross filaments 62 therefore form a “crossed” lasso 76 that facilitates removal of the biological unit. Because the filaments 62 pull each other inward, they tend to not only retain the biological unit in the removal tool more effectively but also may cut the tissue at the distal tip of the tube 64, and subsequently may provide a pulling force to completely sever the biological unit from a surrounding tissue bed. Alternatively, the tension in each filament 62 may be individually adjustable/controllable, similar to the previously described embodiment, and therefore could provide more or less tension and/or allow for more or fewer loops and therefore could be customized to a specific tissue type or condition.

In an alternative configuration, the two filaments may function as conductive (e.g., bipolar RF) elements that surround the tissue. The two filaments may also be conduits to directly pass current through the tissue, or to pass current through the filaments themselves (e.g., nickel-chromium wire), and use them as resistive heating elements to locally cut or sever the tissue.

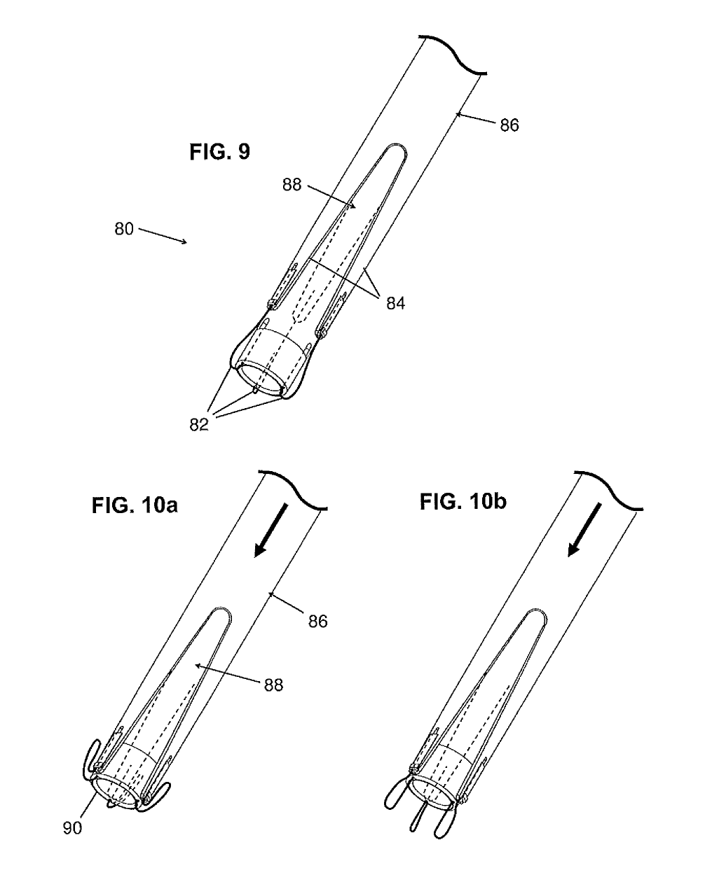

FIGS. 9-12 illustrate another tool 80 for removing biological units that has three circumferentially spaced filaments 82 guided by three tapered fingers 84 on a co-axial member 86 that translates and rotates with respect to an elongated body 88. For the sake of brevity, the constructional details can be assumed to be the same as in the earlier embodiments, although those of skill in the art will understand that numerous variations are possible.

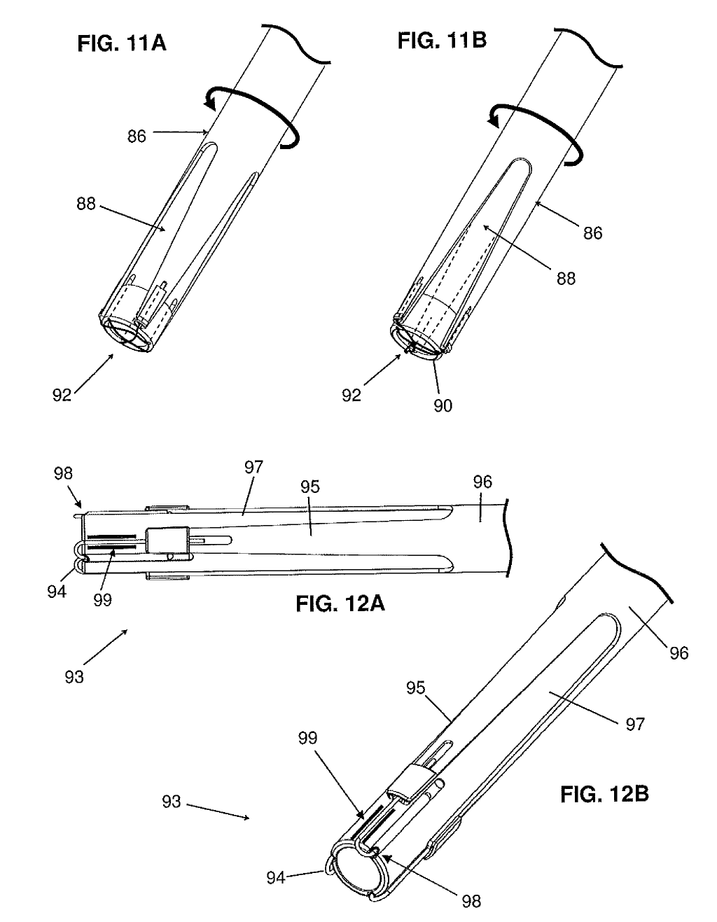

FIG. 11B illustrates the tool 80 after a complete rotation of the co-axial member 86 around the tube or cannula 88. The filaments 82 cross over one another at the distal tip 90 of the cannula 88 and may form a triangular basket or lasso 92. Again, each of the filaments 82 mutually pulls the other filaments inward, and therefore creates an occlusion just beyond the distal end of the lumen of the elongated body/tube 88. The combination of all three filaments 82 can be collectively viewed as an occluding member, and the foregoing discussion will make it clear to those of skill in the art that one or more of such additional filaments can be provided and actuated to constrict about the distal tip 90.

FIGS. 12A-12B illustrate a still further tool 93 for removing biological units that has three circumferentially spaced filaments 94 guided by three tapered fingers 95 on a co-axial member 96 that translates and rotates with respect to an elongated body 97. The filaments 94 are guided by chamfered edge cans or tubules 98 that accommodate filaments in their retracted state, provide relief from wear and better expose a distal cutting edge of the tool during coring of a biological unit. Raised ridges 99 that create channels or recesses may also be provided in the exterior of the elongated body 97 in order to “park” (at least temporarily) the filaments 94 when not in use, such as during coring of a biological unit, to minimize oscillating motion from damaging/disrupting/putting undue tension on the filament(s).

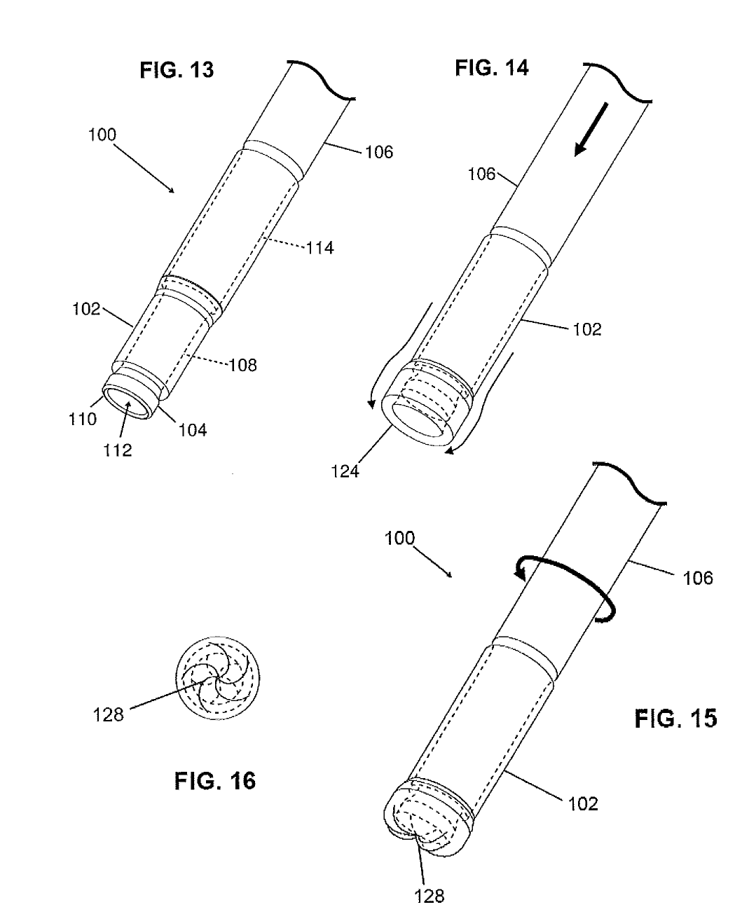

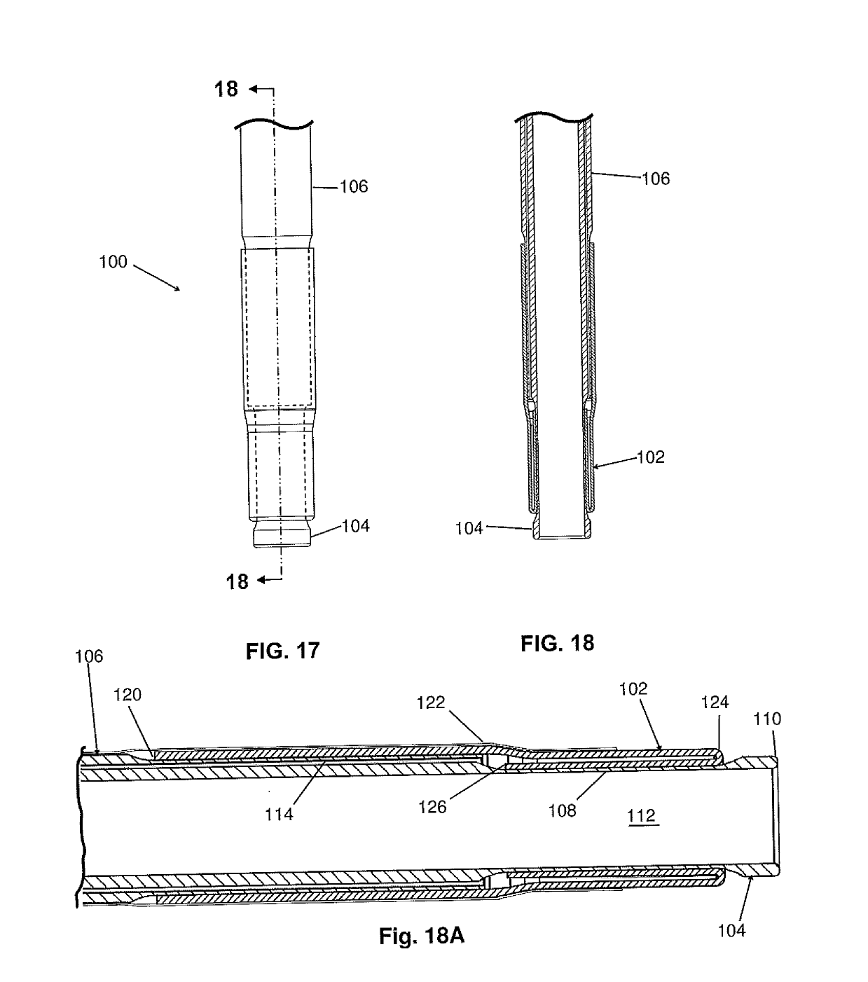

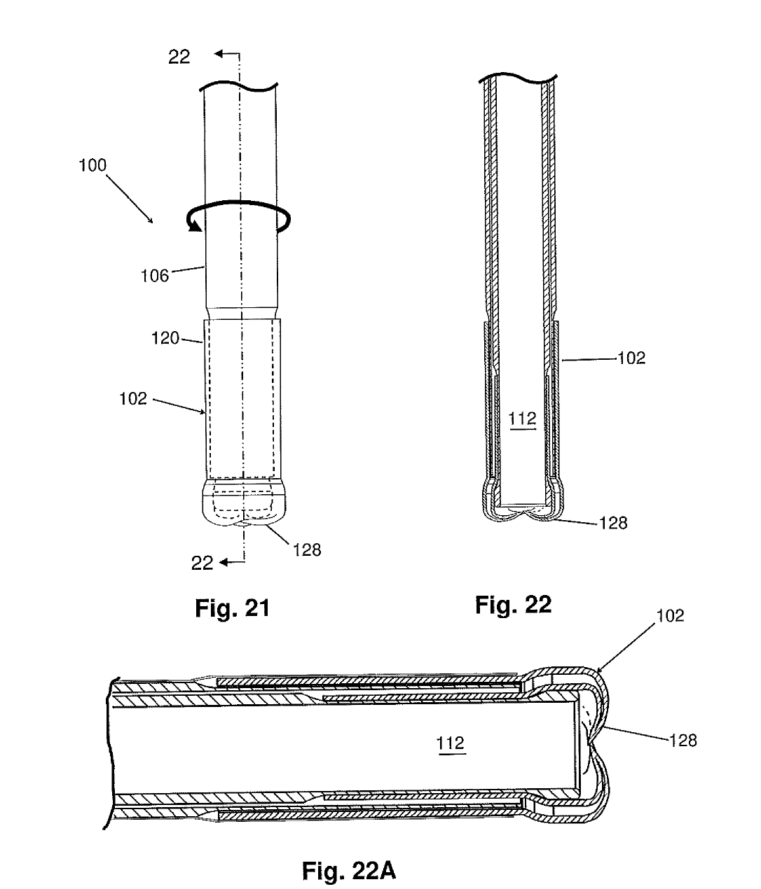

FIGS. 13-16 show a modified biological unit removal tool 100 from those previously described, which instead of filaments utilizes a sleeve-like elastomeric occluding member 102. It should be understood, however, that in certain embodiments, the occluding member 102 may comprise a combination of the elastomeric sleeve and one or more filaments, moreover, in some embodiments, the elastomeric sleeve may be composed of a plurality of filaments. Basic operation of the tool 100 is seen in FIGS. 13-16, while more details are shown in FIGS. 17-22.

The tool 100 has an elongated body or tube 104, and an outer tube or co-axial member 106 with which the occluding member 102 translates and rotates. As seen best in FIGS. 18 and 18A, the tube 104 comprises an elongated body having a relatively constant wall thickness except along a thinned segment 108 just proximal to a distal tip 110. As seen in FIG. 18A, the thinned segment 108 may be formed, for example, by a taper. Alternatively, it may be formed by a step. The distal tip 110 defines an opening leading to a lumen 112. The member 106 likewise has a constant wall thickness except for a thinned distal region 114, which could be formed similarly to the thinned segment 108. Preferably, the tubes 104, 106 are formed of relatively rigid biocompatible material, such as a polymer like Delrin or a metal such as stainless steel, Nitinol, etc. It is worth repeating that the member 106 may be replaced with various co-axial members mounted for rotation about the tube or cannula 104, such as the embodiments with fingers described above.

The occluding member 102, on the other hand, is highly flexible. The occluding member 102, as seen in FIG. 18A, comprises a tubular sleeve, or a balloon, having a proximal segment 120 surrounding the thinned distal region 114 of the member 106. In the illustrated embodiment, the occluding member 102 can be termed an inverted or doubled-over tubular member, and can be made from any suitable elastomer, for example, polyurethane, silicone, Pebax® (Arkema, France), etc. The proximal segment 120 attaches to the member 106 with, for example, adhesive at the thinned distal region 114, or via a thin surrounding heat-shrink sleeve 122, or both. Furthermore, the proximal segment 120 can be affixed at the thinned distal region 114 with an outer metal sleeve (not shown) that is then swaged, crimped, or inwardly deformed to cause a mechanically secure connection. Adhesive may also be used to further reinforce the mechanical interface between any of the mating surfaces. The sleeve 122 extends from the member 106 over a substantial portion of the occluding member 102 so as to provide a connection therebetween sufficient to transfer axial pulling and pushing forces. The occluding member 102 extends distally past the member 106 and folds inward upon itself at fold 124 along the length of the thinned segment 108 of the tube 104. An inner layer 126 of the occluding member 102 may be attached to the thinned segment 108 through the use of adhesive, heat-shrink sleeve, or crimped metallic tube, or in any combination, or the like. Consequently, the occluding member 102 has a first end (the proximal segment 120) affixed to the member 106, and a second end (the inner layer 126) affixed to the tube 104. In one embodiment of the invention, an inner sleeve (not shown) may be incorporated to prevent the occluding member 102 from “peeling back” at the distal end of the tool in operation, and in particular after repetitive use. The inner sleeve may be in the form of a thin surrounding heat-shrink sleeve, a tubular sleeve or ring, formed of relatively rigid biocompatible material, such as a polymer like polyester or a metal such as stainless steel, Nitinol, etc. The inner sleeve is disposed in the region between the portions of the occluding member 102 that fold over one another, but not necessarily along the entire length of the folded over portions. A simple ring disposed at the distal end, at the fold 124 would suffice.

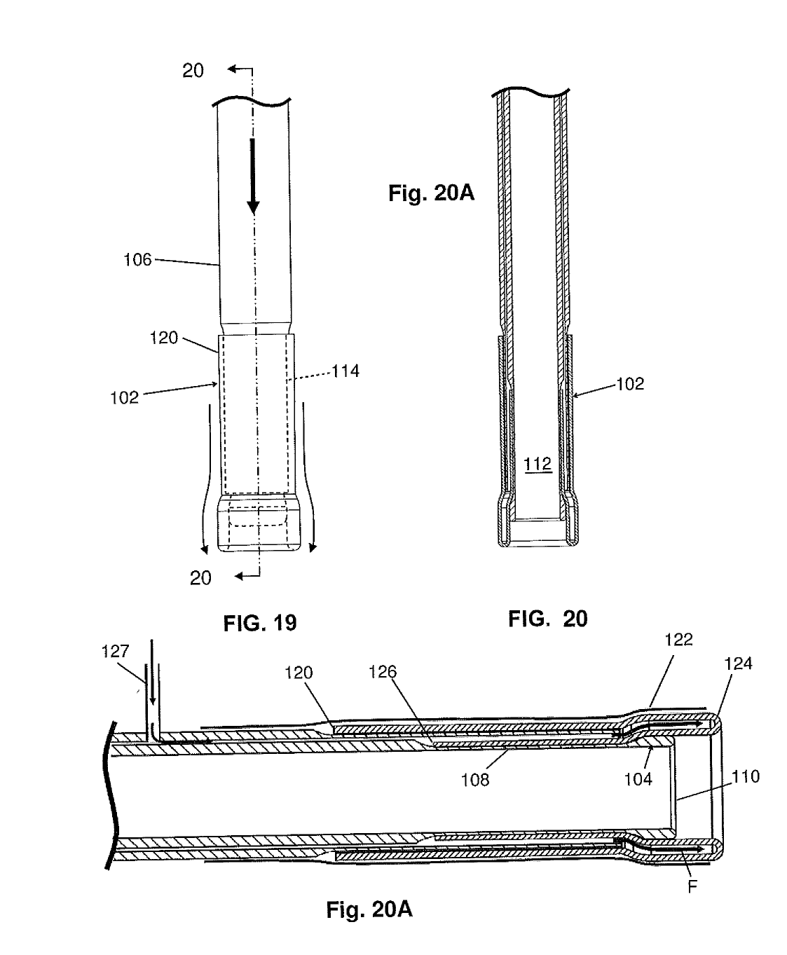

Although not shown, the inner tube 104 and outer or co-axial tube or member 106 are mounted for relative axial and rotational motion. More particularly, the inner tube 104 is desirably held stationary while the co-axial member 106 slides linearly and rotates thereover, however, the alternative configuration where the inner tube 104 slides and rotates within the stationary co-axial member 106 is also within the scope of the present invention. FIGS. 19-20A show the member 106 advanced distally over the tube 104 relative to the corresponding views of FIGS. 17-18A. FIG. 20A best illustrates how far the member 106 advances, which causes the rolling fold 124 of the occluding member 102 to translate distally past the distal tip 110 of the tube 104. In the illustrated embodiment, the member 106 slides distally until just before the heat-shrink sleeve 122 reaches the fold 124. At this point, the dual-layer occluding member 102 may extend approximately 0.001-5.0 mm, and in some situations between 0.1-1.0 mm, past the distal tip 110. It is even possible that the occluding member 102 ends up being flush with the distal tip 110, and its material properties permits it to stretch over the distal tip upon being twisted to form the occlusion described below.

FIG. 20A also illustrates an optional feature of the biological unit removal tool 100 in which compressed air or fluid, such as saline is introduced to help deploy the flexible occluding member 102. In one embodiment, a sealed side port 127 opens to a channel or annular space between the inner tube 104 and co-axial member 106. Fluid or air passes distally and enters the annular region between the proximal segment 120 and inner layer 126 of the occluding member 102, adjacent the rolling fold 124, as seen by flow arrows F. The pressure and lubrication of such flow helps extend the rolling fold 124 as the co-axial member 106 moves forward and backward over the inner tube 104 (or visa versa). Alternatively, in some embodiments the co-axial member 106 may not need to advance/retract over the tube 104 because the fluid or gas could “balloon” a stretchable or loose occluding member 102 out when pressurized and retract it when depressurized. In those embodiments, the step of advancement of the co-axial member 106 over the tube 104 (or retraction of the tube 104 into the lumen of co-axial member 106 to achieve the same) may be omitted.

FIGS. 16 and 21-22A show an example of a biological unit removal tool 100 with the occluding member 102 deployed over the distal tip 110. That is, after reaching the extended configuration shown in FIG. 20A, the tube 104 and the co-axial member 106 are rotated relative to one another to cause a distal portion 128 on the occluding member 102 to substantially narrow or close over the distal tip 110. Desirably, the member 106 rotates while the tube 104 remains stationary, although the opposite may occur. Because the member 106 is affixed to the proximal segment 120 of the occluding member 102, and the tube 104 is affixed to the inner layer 126, relative rotation of the concentric tubes exerts torsional forces along the occluding member 102. The distal portion 128 may be termed an “iris portion” because it constricts over the distal tip 110 much like the iris in an eye. Closure of the iris portion 128 results because of the absence of inner support in the area of the rolling fold 124. That is, the torsional stress exerted along the occluding member 102 is at least partially transferred to the area of the unsupported rolling fold 124 (and causes the region of the unsupported rolling fold 124 to inwardly deform due to the twisting of the occluding member 102).

The occluding member 102 therefore undergoes a transition from lying outside of the lumen 112 at the distal tip 110, and occluding the opening to the lumen 112 at least partially. The iris portion 128 may intimately engage with the tissue, and further torsion applied may cause sufficient constriction (so as to exceed the local strength of the tissue) for tissue to be severed/separated/disengaged from its connected portion or tissue bed, so as to effectively achieve a cutting type action. In the illustrated embodiment, the iris portion 128 closes completely, although the benefit of the constricting iris may be realized without a complete closure. For example, the iris portion 128 may narrow the diameter of the lumen 112, for example, by 50% and still provide sufficient retention force (or traction) on the biological unit to help disengage it from the surrounding tissue bed.

As mentioned above, the various biological unit removal tools of the present invention may be operated using manual, semi-automated, fully-automated, including robotic systems. The principles described herein may be incorporated into a variety of such systems, and are particularly useful in follicular unit harvesting systems, whether they are manual or automated.

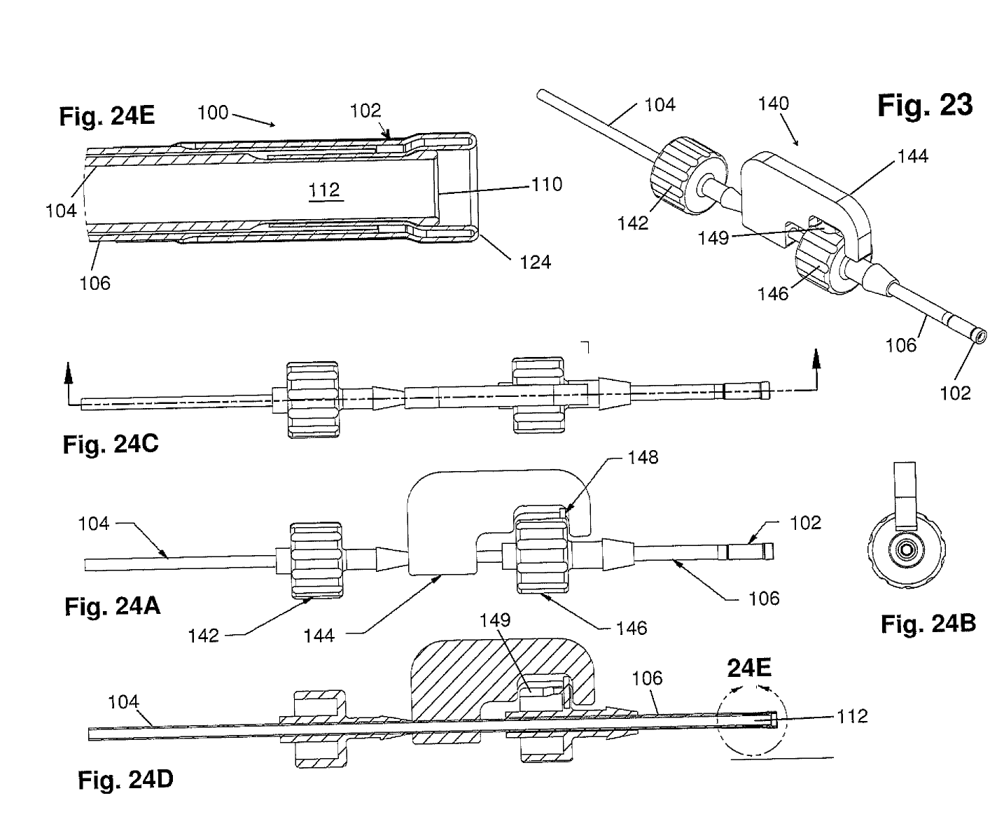

For example, FIGS. 23 and 24A-24E illustrate an example of a hand-held system 140 for operating a biological unit removal tool similar to that shown in FIGS. 13-22, and certain elements will be given the same numbers. Indeed, the detailed view of FIG. 24E is very similar to that of FIG. 20A, and illustrates the distal end of the removal tool 100 with the occluding member 102 extending between and covering the ends of the elongated body 104 and the co-axial member 106.

As seen in FIG. 24D, the tube 104 extends the length of the system 140, defining a continuous lumen 112 therethrough. A quick turn coupling 142 and a guide bracket 144, as best seen in FIG. 23, mount onto the exterior of the tube 104. The member 106 commences within a second quick turn coupling 146. The member 106 concentrically fits closely over the tube 104 and is permitted to slide axially and rotate thereover. A small dowel pin 148 provides a mechanical stop that shows the user that a final position has been reached, and also protects against accidental twisting in the other direction.

An operator can easily hold in his/her hands the system 140, which in certain embodiments may have a length of between 10-200 mm, and more specifically between 20-100 mm and manipulate the occluding member 102 and the concentric tubes 104, 106. More particularly, the second quick turn coupling 146 and the member 106 are shown advanced with respect to the first quick turn coupling 142 and guide bracket 144 such that the rolling fold 124 of the occluding member 102 extends past the distal tip 110 of the tube 104. Relative rotation between the first and second quick turn couplings 142, 146 will cause the occluding member 102 to form the iris portion 128, as seen in FIGS. 15 and 16. By retracting proximally the second quick turn coupling 146 relative to the guide bracket 144, the rolling fold 124 is also retracted in order to achieve the configuration as shown in FIG. 18A. In this retracted position, the first quick turn coupling 146 has a groove 149, as seen in FIGS. 23 and 24D, that accommodates the guide bracket 144 in order to prevent unwanted rotation of the occluding member 102.

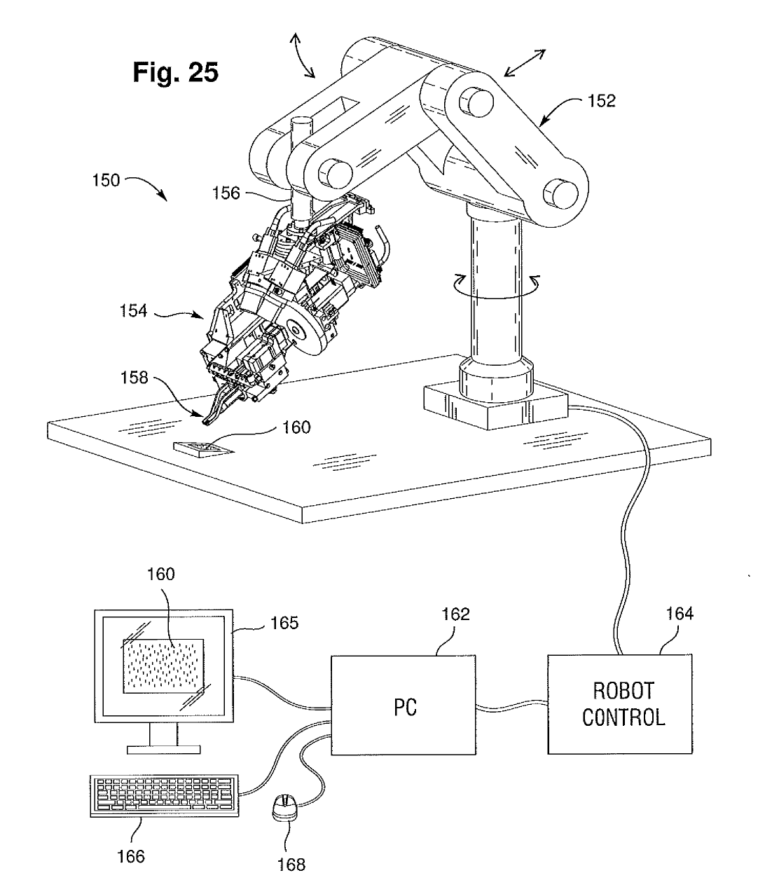

FIG. 25 is a schematic perspective view of an example of a robotic system 150 for operating biological unit removal tools of the inventions described herein. The system 150 includes a robotic arm 152 having a head assembly 154 mounted for rotation on a down tube 156 of the robotic arm. Various arrows are shown to illustrate the movement capabilities of the system 150. Furthermore, as will be seen below, motors and other such movement devices incorporated in the head assembly 154 enable fine movements of an operating tip 158 in multiple directions. The operating tip 158 represents any of the biological unit removal tools described herein.

The operating tip 158 is shown positioned over a body surface 160, in this case a strip of tissue having hair follicles thereon. Personal computer 162 acting, for example, through a robotic control 164 controls the various movement devices of the robotic arm 152 and head assembly 154. An operator monitors conditions and provides instructions through a monitor 165, keyboard 166, and mouse 168. A magnified image of the body surface 160 can be seen on the monitor 165.

A sequence of operation of a dual-cannula biological unit removal tool 170 using an automated system like robotic system 150 in FIG. 25 is shown in FIGS. 26A-26C. The operating tip 158 includes the biological unit removal tool 170 held within a collet 172. The collet 172 holds (and moves) a co-axial member 174 co-axially arranged about an intermediate cannula 176, which in turn is held and moved by another collet not shown in the figures. An inner cannula 178 slides and desirably rotates within the intermediate cannula 176, and is also separately moved by another collet not shown in the figures. As was previously explained, the co-axial member 174 may be a tube or a cannula co-axially positioned relative to the intermediate tube or cannula 176. Those of skill in the art will understand various ways to manipulate two cannulas 178 and 176 and co-axial member 174 with respect to one another, typically by terminating the cannulas/member at different lengths, as was seen with the example of a manual system embodiment of FIGS. 23-24E. The cannulas, for example, could be controlled with electric servo or stepper motors, linear actuators, solenoids, hydraulics and/or pneumatics using any combination of gears, belts, chains, or any other drive system.

In one embodiment, the inner cannula 178 has a sharpened distal end 180 that is thrust at high speed through the intermediate cannula 176 to pierce a body surface BS to a short depth. The intermediate cannula 176 and co-axial member 174 may then be advanced together or sequentially to force an occluding member 182 through body surface BS and surround, for example, a follicular unit FU. The inner cannula 178 may pierce the tissue to an initial depth of about 1 to 2 mm, while the intermediate cannula 176 may be a blunt dissection cannula that may dissect through the cutaneous and subcutaneous tissue to a farther depth of about 5-7 mm. In some embodiments of the method, the distal end of the intermediate cannula 176 may extend past the follicular unit FU as seen in FIG. 26B to make sure that the bulb of the follicular unit is captured. The user then actuates the occluding member 182 to close an iris portion 184, as seen in FIG. 26C, and the cannulas/tubes/members 174, 176, 178 are withdrawn from the body surface BS, thus capturing the follicular unit FU. However, in alternative embodiments (not shown), the intermediate cannula 176 may simply extend to a position along the length and around the follicular unit so that when the iris portion 184 closes and intimately engages the follicular unit, it assist in frictionally pulling it out without tearing or damaging the follicular unit.

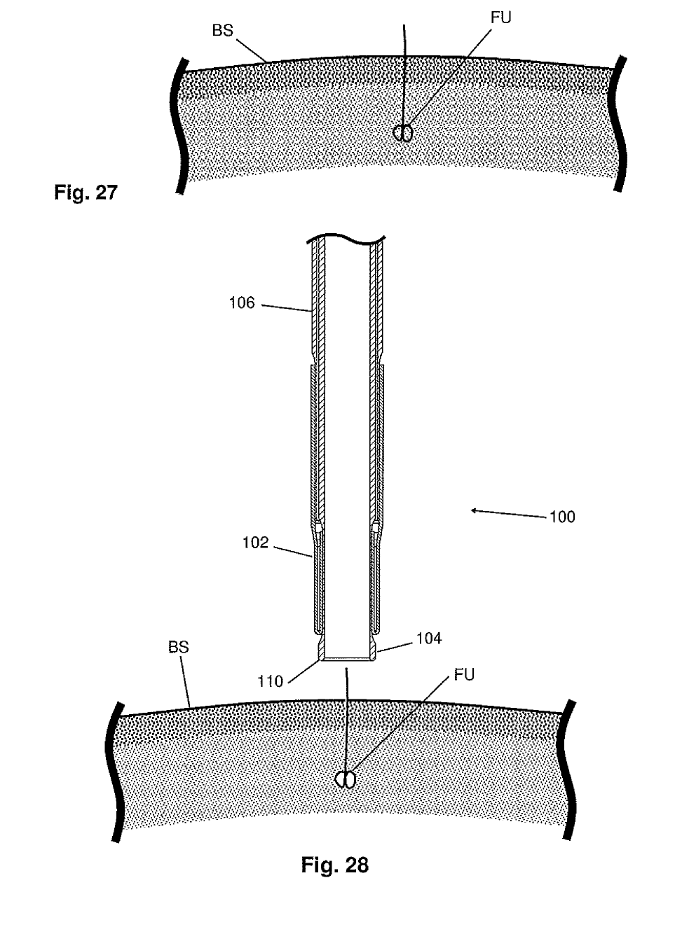

FIGS. 27-31 more deliberately illustrate the biological unit removal tool 100 of FIGS. 13-22 in operation removing a follicular unit FU from a body surface BS. FIG. 28 shows advancement of the tool 100 with the co-axial member 106 and occluding member 102 retracted to expose the distal tip 110. The distal tip 110 may be sharpened or include cutting teeth to facilitate puncture into the body surface BS, or alternatively, the inner cannula 178 (not shown) has already pierced the body surface BS to an initial depth, as described previously.

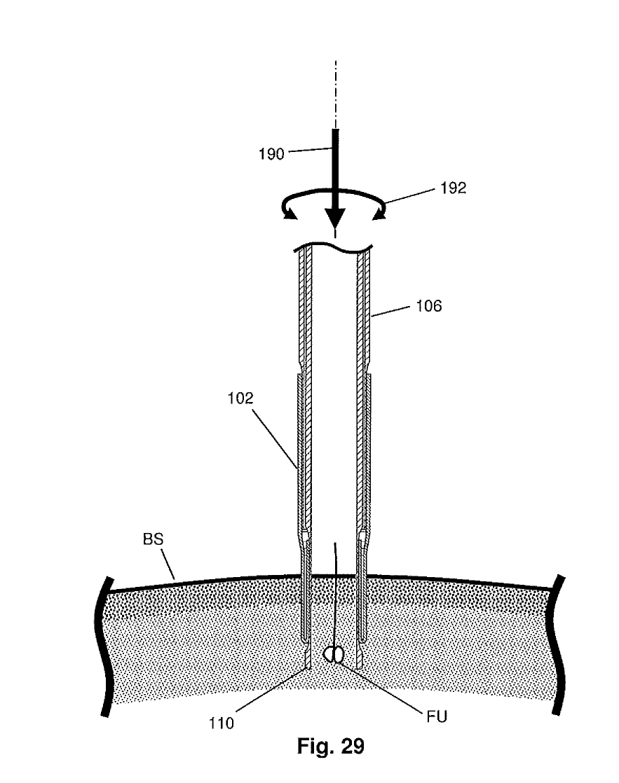

FIG. 29 shows the removal tool 100 after entering the body surface BS in the direction of arrow 190 such that the distal tip 110 extends, for example, approximately as deep as the bulb of the follicular unit FU. The thinned segment 108 of the tube 104 just proximal to a distal tip 110 provides an inset so that the double layer occluding member 102 in a region does not substantially increase the diameter of the tool 100 proximal to the distal tip. Desirably, the thinned segment 108 extends a sufficient distance to extend to the illustrated depth at or just beyond the bulb of the follicular unit FU in those embodiments where closure of the occluding member beyond the bulb is desired. The rotational arrow 192 indicates that the entire tool 100 may be rotated during the step of puncturing and advancing through body surface BS.

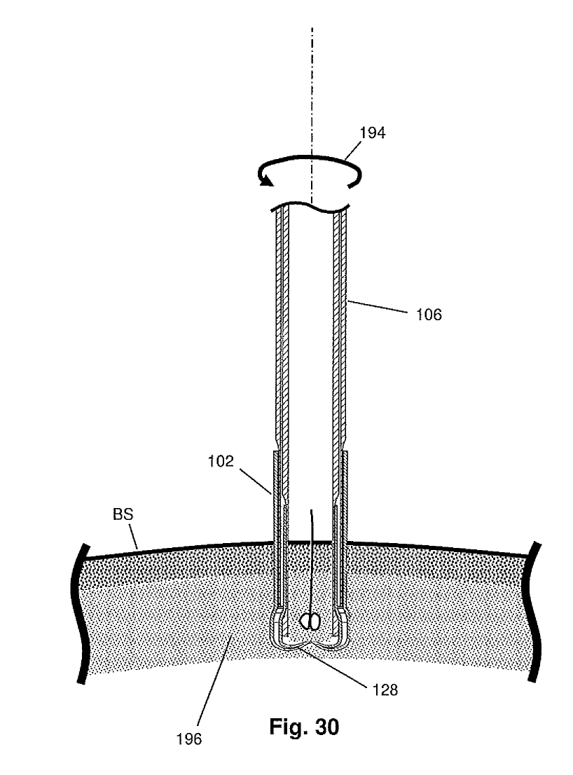

FIG. 30 shows deployment of the occluding member 102 at the distal end of the removal tool 100. That is, rotational arrow 194 indicates rotation of the co-axial member 106 relative to the elongated body or tube 104, or in general relative rotation therebetween. As explained above, the occluding member 102 forms the iris portion 128 because of the torsional forces therein. The iris portion 128 constricts inward below the follicular unit FU as shown in FIG. 30, or alternatively, as previously explained it may constrict circumferentially around the follicular unit FU somewhere along its length as long as it does not damage it. In certain cases, the tissue between the follicular unit and the surrounding tissue bed 196 may be severed, although, in some cases very tightly clamping on without severing the tissue may be sufficient.

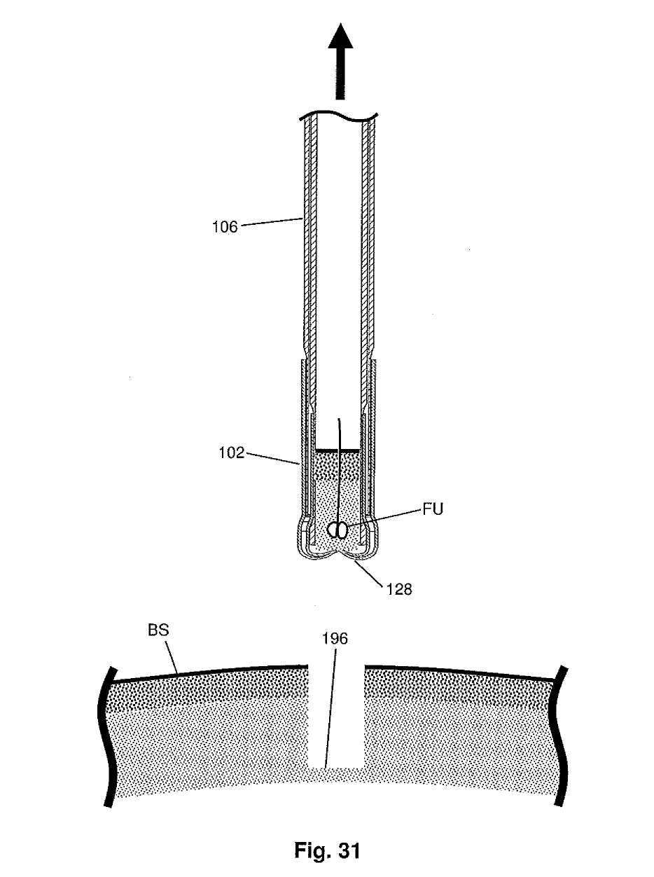

Subsequently, FIG. 31 illustrates axial removal of the tool 100 with the follicular unit FU retained within the lumen 112. Suction within the lumen 112 may be applied in conjunction with the severing and retention properties of the iris portion 128 to cleanly remove the follicular unit FU from the body surface BS. Although not shown, the follicular unit FU can then be expelled distally from the tool 100 using a positive pressure or solid obturator, or the follicular unit may be pulled through the tube 100 into a storage container or cartridge.

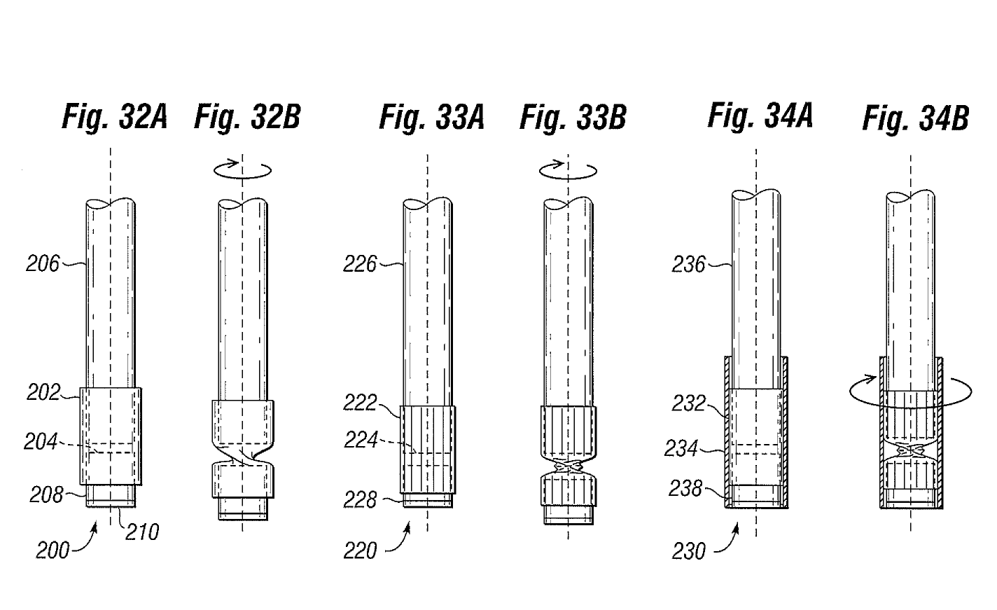

FIGS. 32A and 32B illustrate a still further biological removal tool 200 having a tubular occluding member 202 positioned to constrict inward through axial gaps 204 between two co-linear tubular members 206, 208. Alternatively, it could be described as axial gap(s) 204 positioned along the length of one removal tool 200 separating the removal tool in 2 or more co-linear portions. The axial gap 204 could also be large enough to encompass the entire biological specimen so as to circumferentially constrict along the length of the biological specimen without damaging the specimen, but instead to provide substantial traction along the length of the specimen. Furthermore, the inner surface of the tubular occluding member 202 can have ridges, barbs, knobs, cones, or any other surface feature that enhances traction with the tissue of the biological specimen. The occluding member 202 is desirably a solid elastomeric sleeve that constricts inward into the aligned lumens of the tubular members 206, 208 due to torsional forces therein. Relative rotation of the two tubular members 206, 208 may be accomplished by holding one of the two stationary while rotating the other, or the distal tubular member 208 may be provided with small barbs or texturing to grips tissue surrounding the biological unit being removed. That is, the distal tubular member 208 may include a sharp distal tip 210 as well as the aforementioned gripping means such that rotation of the proximal tubular member 206 causes constriction of the member 202.

FIGS. 33A and 33B illustrate another alternative biological removal tool 220 having a fibrous tubular occluding member 222 positioned to constrict inward through axial gaps 224 between two co-linear tubular members 226, 228. Rather than a solid wall construction, the occluding member 222 comprises a series of axially-oriented fibers that collapse inward into the gaps 224 from portion in the occluding member. As with the embodiment of FIGS. 32A-32B, relative rotation between the tubular members 226, 228 may be caused by holding one stationary and rotating the other, or by providing barbs or some anchoring feature on the distal tubular member 228.

FIGS. 34A and 34B illustrate a still further alternative biological removal tool 230 having a tubular occluding member 232 surrounded by a sleeve 234 and positioned to constrict inward through axial gaps between two tubular members 236, 238. The tool 230 is similar to the tool 200 in FIGS. 32A-32B, with the addition of sleeve 234. The sleeve 234 is desirably affixed to the distal member or portion 238 and facilitates relative rotation between the tubular members/portions and can be controlled manually or through a robotic mechanism.

In addition to the embodiments described above, the present application contemplates biological removal tools in which an occluding member resides within a removal cannula. For example, the occluding member may be formed of a helical spring-like element positioned within the cannula that constricts inward when twisted to reduce a lumen of the cannula. The coils of the spring-like occluding member extend closely along an inner lumenal wall of the cannula, and anchor therein to prevent distal movement. A proximal end of the occluding member may be rotated relative to the surrounding cannula to tighten the coils, thus narrowing a throughbore in the occluding member.

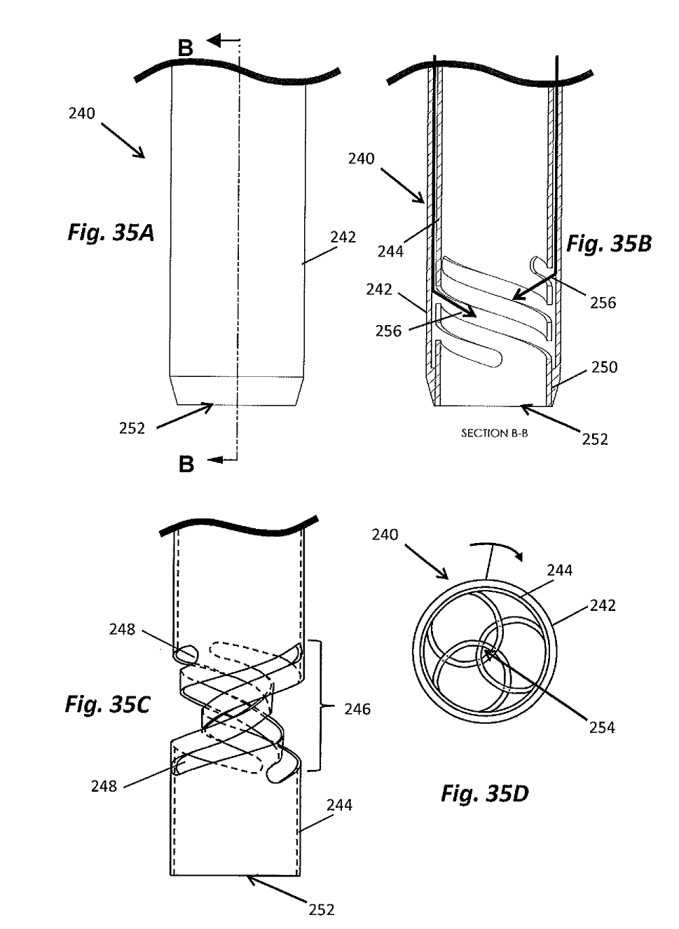

A further alternative biological unit removal tool may function the same as the spring-like occluding member but instead comprising a tubular member with helical openings cut therein. Again, the tubular member extends closely along an inner lumenal wall of the cannula and affixes thereto with a weld or similar expedient. The tubular member constricts inward upon twisting to reduce a lumen of the cannula. One such example is illustrated in FIGS. 35A to 35D where a biological removal tool 240 comprises an outer elongated body or tube 242, and an inner tubular occluding member 244 with helical openings cut therein. The occluding member 244 is an inner elongated body that is disposed co-axial to the outer elongated body 242, and is configured and positioned such that the elongated body 242 and the occluding member 244, or at least an occluding portion 246 of the occluding member 244, are able to rotate relative to each other. The occluding member 244, or at least the occluding portion 246 of the occluding member 244, comprises a series of helical openings or slots 248 that are arranged in a spiral configuration around the circumference and down the axial length of the occluding portion 246, such that the slots 248 essentially form a helical would feature along the axial length of occluding portion 246. In one preferred embodiment, the slots traverse at least 180 degrees around the circumference of the occluding member 244, however, it is not required. Also, in some embodiments one or more slots 248 are located in proximity to the distal end of the tool 240. Each slot 248 may comprise a single slot or a plurality of adjacent slots, for example, three slots as can be seen in FIG. 35D. The slots 248 may be formed by cutting (such as by laser ablation) or welding, they may be molded, or otherwise formed in the occluding member 244 and have a shape other than that depicted in the FIGS. 35B-C. Care should be taken to ensure, however, that in forming the slots 248, the overall structure of the occluding member 244 is not unduly weakened, and that it may still function adequately in the follicular unit removal process. The number, width and pitch of the slots 248 should be carefully determined, depending upon, for example, the material being used, the thickness of the structure, the intended use of the removal tool, and other considerations that will be apparent to one skilled in the art. It will be apparent that if the slot width is too narrow, rotational movement will be limited. The shape, size, number, longitudinal positioning and configuration of the slots 248 may vary along the same occluding member 244. The slots 248 need not be positioned symmetrically about any axis of the tool 240. FIG. 35B is a cross-section taken on B-B of FIG. 35A, and shows one example where the distal end of the outer elongated body 242 may be attached to the distal end of the occluding member 244 at 250. This attachment 250 can be formed by the use of adhesion, crimping, welding or any other known suitable attachment method. In the example of FIG. 35B, the attached distal ends of the elongated body 242 and the occluding member 244 define an opening into a lumen 252.

FIGS. 35C and 35D illustrate the tool 240 after the outer elongated body 242 has been rotated. Though shown as rotating clockwise relative to the reader, rotation may be anti-clockwise. If the tool 240 is a hand-held tool, the rotation may be manual, or it may be performed by a semi-automatic or automatic means, utilizing a solenoid or a motor, for example. If the tool 240 forms part of a robotic or automated system, semi-automatic or automatic mechanism or feature to initiate rotation may be employed. Rotation of the outer elongated body 242 relative to the occluding member 244 allows the width of the slots 248 in the portion 246 of the occluding member 244 to be reduced. However, since the occluding member 244 is attached at least via attachment 250 at the distal end 252 of the tool 240, this attachment 250 prevents the occluding member from shortening in axial length at this end, and causes at least the portion 246 of the occluding member 244 to twist and compress radially inwards. Consequently, the radially inward movement of all or at least part of the portion 246 of the occluding member 244 is such that it protrudes into the lumen and constricts, or in some cases closes, the lumen that was previously substantially clear. In this configuration, at least a part of the portion 246 is able to clamp any follicular unit that may be disposed within the lumen of the tool 240 by closing of or constricting (fully or partially) the gap 254. It will be apparent that in some embodiments the occluding member 244 may be attached to the elongated body 242 at the proximal end of the tool 240, or at both ends. The tool 240 then can be retracted to remove the follicular unit from the body surface. When the outer elongated body 242 is rotated back to its original position, the slots are allowed to return to their initial width and configuration, and the portion 246 of the occluding member 244 expands radially outwardly such that once again it is in-line with the other portions of the occluding member 244 that do not have slots. Additionally, portion 246 of the occluding member 244 can be further expanded radially, such that it is intimate with the inner surface of the elongated body 242. This further ensures that the opening for portion 246 of the occluding member 244 is maximized in order to minimize exposed edges that may catch fragments of tissue, etc. Any follicular unit that was retained by the portion 246 of the occluding member 244 is then released, and the lumen is substantially clear again, ready to receive another follicular unit. This configuration enables the lumen of the elongated body 242 to be kept substantially open when follicular units are not received. It will be apparent to those skilled in the art, that although this embodiment has been described with the outer elongated body 242 being rotated to cause the portion 246 of the occluding member 244 to at least partially occlude the lumen of the tool 240, the same result may be attained by rotational movement of the occluding member 244 relative to the outer elongated body 242, or by the relative rotational motion of both the outer elongated body 242 and the occluding member 244. It is not required, however, that any of the distal or proximal ends (or first or second ends) of the occluding member 244 be attached to the elongated body, and occlusion of the lumen of the elongated body may be achieved simply by rotation of either the first end, or the second end, or both ends of the occluding member 244 relative to each other.

Optionally, fluid, such as saline may be introduced to help flush, minimize or prevent tissue/debris build-up after multiple follicular unit collections. Fluid introduction may also improve the lubricity and help elevating and retaining the follicular unit in the lumen of the harvesting tool 240. Irrigation could be introduced, for example, in the spacing between the outer elongated body 242 and the occluding member 244 as illustrated by the arrows 256 in FIG. 35B.

In certain embodiments, it may be advantageous to use a spring-loaded mechanism for twisting/rotation the coaxial member relative to the inner tube. For instance, for harvesting follicular units, once deep into the body surface and past the hair bulbs, the filaments may be quickly deployed using a spring. A thin filament (e.g. nitinol wires), thus can provide more of a cutting action because the tissue would have less time to accommodate/adapt to the constricting force of the wires. More generally, the filaments or elastomeric occluders may be opened and closed at different rates for certain types of tissues. The devices can be actuated rapidly or slowly, or the rate can be varied, by increasing, decreasing during an opening or closing cycle, or oscillated cycling (repeated opening and closing to provide a “scissoring effect”). More rapid deployment will provide better tissue “cutting.”

A still further feature that could be used with various embodiments involves a mechanism for controlled/adjustable constriction level or twisting magnitude. This would enable better tuning of the device to ensure the right amount of rotation needed on the fly in order to tune the devices for optimal retraction and/or cutting of different types of biological samples, including follicular units. For instance, the level of rotation for the filament concepts needs to be tuned in to prevent filament breakage, and adding a feature that minimizes over-tightening may be advantageous. For instance, the small dowel pin 148 shown in FIG. 24D provides a mechanical stop that shows the user that they have reached the final position and also to protect against accidental twisting in the other direction. Alternatively, controlled/adjustable rotation could be achieved using markers on the sides of concentric tubes to gauge relative rotation, or a notch, or a knob that is indexed to indicate various amounts of relative rotation. An audible clicking device could be added, for instance, to signal 30° rotation increments.

If the actuation is robotic, then the robotic mechanism can be programmed to provide the right amount of rotation (it could be done, for example, based on testing). A preferred rotation range may be anywhere between 1-1800° (5 full turns), or more likely 90-720°. One methodology would be to measure the amount of torque imparted on a follicular unit FU during a constriction cycle or series of test cycles (using a load cell to measure torque), to determine a certain maximal level that can be set and not exceeded in order to prevent device failure, especially for the filaments.

In certain embodiments, it may be desirable to provide a mechanism for overload protection of the occluding member, especially when it is in a form of filaments to prevent filament breakage due to overload. For example, a small coiled spring element or a small elastomeric column on the proximal portion of a can that contains the filaments can be used as a safety mechanism that will allow for additional displacement without failure. Additionally, the superelastic properties of nitinol wire may be utilized to also provide a similar safety feature by allowing for a certain amount of “overload” displacement inherent in the material itself. Furthermore, filaments could be designed using a metallic (e.g. Nitinol) coil-covered material like suture, Kevlar, etc. to provide a robust and protected exterior while at the same time having the strength and flexibility of a multi-filament material. Another protection mechanism for the filaments would be to have a torsional spring in series with the actuation mechanism for the outer co-axial tube or member in order to provide a certain amount of displacement and therefore overload protection. The spring-type could also be a constant-force spring, so that the force level remains the same despite the displacement.

While the invention has been described in its preferred embodiments, it is to be understood that the words which have been used are words of description and not of limitation. Therefore, changes may be made within the appended claims without departing from the true scope of the invention. By way of non-limiting example, it will be appreciated by those skilled in the art that particular features or characteristics described in reference to one figure or embodiment may be combined as suitable with features or characteristics described in another figure or embodiment.

What is claimed is:

1. A biological tissue removal tool, comprising:
an elongated body having a lumen sized to receive a biological unit and a distal tip configured to penetrate a body surface; and
an occluding member disposed coaxially with the elongated body and having a first end and a second end, the occluding member having a first configuration where the lumen of the elongated body is substantially open, and a second configuration where at least a portion of the occluding member partially or fully occludes the lumen of the elongated body,
wherein in the second configuration, at least one of the first end or the second end of the occluding member is rotated relative to the other.

2. The tool of claim 1, wherein the elongated body comprises a first tube, and the tool further includes a second tube, wherein the occluding member connects at the first end to the first tube and at the second end to the second tube.

3. The tool of claim 2, wherein the first and second tubes are concentrically arranged as inner and outer tubes, respectively, and the occluding member connects to an exterior of both tubes.

4. The tool of claim 2, wherein the occluding member in the second configuration constricts over the distal tip of the first tube.

5. The tool of claims 1, wherein the occluding member comprises one or more of a flexible sleeve, a helical slot, a suture, a thread, a strand, a wire, a string, a fiber, a cable, a coil, a yarn, or a filament.

6. The tool of claim 3, wherein the occluding member is a flexible sleeve attached on the first end to a thinned segment proximal to the distal tip of the inner tube and on the second end to a thinned region on the outer tube, and the first and second ends extend in a proximal direction such that the occluding member folds back upon itself and forms a rolling fold at a distal end thereof that can be advanced beyond and constrict over the distal tip of the inner tube.

7. The tool of claim 2, wherein the first and second tubes are co-linear and spaced apart across an axial gap, and the occluding member in the second configuration constricts into the lumen through the axial gap.

8. The tool of claim 1, wherein the occluding member occludes the lumen of the elongated body proximally away from the distal tip of the elongated body.

9. The tool of claim 1, wherein the occluding member comprises at least one filament with a first end of the filament fixed with respect to the elongated body and a second end of the filament configured to rotate around the elongated body, the filament being arranged to transition upon rotation around the elongated body from a position in the first configuration generally outside of the lumen of the elongated body to a position in the second configuration where at least a portion of the filament extends across the lumen at the distal tip of the elongated body.

10. The tool of claim 9, further comprising a channel structured to accommodate the at least one filament in the first configuration.

11. The tool of claim 9, wherein the occluding member comprises a plurality of filaments each with the first and second ends, wherein in the second configuration the filaments extend across the lumen at the distal tip of the elongated body in an overlapping fashion.

12. The tool of claim 5, wherein the occluding member comprises an occluding portion having a plurality of helical slots, and wherein at least one of the first end or the second end of the occluding member is fixed with respect to the elongated body, and upon relative rotation of at least the occluding portion and the elongated body the occluding portion of the occluding member occludes the lumen of the elongated body.

13. The tool of claim 1, further comprising one or more of a mechanism for controlled rotation and/or controlled tension of the occluding member, or an overload protection mechanism.

14. The tool of claim 1, wherein the elongated body and the occluding member are axially movable relative to each other.

15. The tool of claim 1, wherein the tool is configured to be operatively connected to a robotic arm.

16. The tool of claim 1, wherein the biological unit is a follicular unit and the biological tissue removal tool is a hair harvesting tool.

17. A follicular unit harvesting tool, comprising:
an elongated body having a lumen sized to receive a follicular unit and a distal tip configured to penetrate a body surface;
a co-axial member mounted on the elongated body; and
an occluding member having a first end attached to the elongated body and a second end attached to the co-axial member,
wherein the occluding member is configured to at least partially occlude or close the lumen of the elongated body upon relative axial and/or rotational movement of the elongated body and the co-axial member.

18. The tool of claim 17, wherein the occluding member comprises at least one filament with a first end and a second end, the at least one filament being arranged to transition upon relative rotation of the co-axial member and the elongated body from a position substantially adjacent the elongated body to a position where a portion of the filament extends across the distal tip of the elongated body.

19. The tool of claim 17, wherein the occluding member comprises a plurality of filaments, wherein in an occluding position the filaments extend across the distal tip of the elongated body in an overlapping fashion.

20. The tool of claim 17, further comprising an overload protection mechanism for the occluding member.

21. The tool of claim 17, wherein the elongated body comprises two co-axial inner and outer harvesting cannulas, wherein the co-axial member is mounted on the outer harvesting cannula.

22. The tool of claim 17, wherein the co-axial member comprises a second elongated body axially movable with respect to the first elongated body, and the occluding member comprises a tubular member connected to distal ends of both the first and second elongated bodies.

23. The tool of claim 22, wherein the tubular member is folded back upon itself and forms a rolling fold at a distal end thereof that can be advanced beyond a distal tip of the first elongated body.

24. The tool of claim 22, wherein the tubular member is a flexible sleeve.

25. An automated system for harvesting follicular units from a donor area, comprising:
a moveable arm;
a hair harvesting tool operably connected to the moveable arm, the harvesting tool comprising:
an elongated body having a lumen sized to receive a biological unit and a distal tip configured to penetrate a body surface; and
an occluding member disposed coaxially with the elongated body and having a first end and a second end, the occluding member having a first configuration where the lumen of the elongated body is substantially open, and a second configuration where at least a portion of the occluding member partially or fully occludes the lumen of the elongated body,
wherein in the second configuration, at least one of the first end or the second end of the occluding member is rotated relative to the other; and
a control mechanism for controlling movements of one or more of the moveable arm or the harvesting tool.

26. The system of claim 25, wherein the moveable arm is a robotic arm.
